(19) United States
(12) Patent Application Publication
(10) Pub. No.: US 2025/0381135 A1
(43) Pub. Date: Dec. 18, 2025

(54) HYDROGELS COMPOSED OF BINARY COPOLYMERS AND APPLICATIONS THEREOF

(71) Applicant: AESCULATECH, INC., Los Angeles, CA (US)

(72) Inventors: Roby Menefee, Los Angeles, CA (US); Niki Bayat, Los Angeles, CA (US)

(21) Appl. No.: 19/240,222

(22) Filed: Jun. 17, 2025

Related U.S. Application Data
(60) Provisional application No. 63/661,185, filed on Jun. 18, 2024.

(51) Int. Cl. A61K 9/06; A61K 9/00; A61K 31/785

(52) U.S. Cl. CPC A61K 9/06; A61K 9/0048; A61K 31/785

(57) ABSTRACT

Described herein are hydrogels composed of binary copolymers. In one aspect, the copolymer comprises at least one residue from (a) an acrylamide selected from the group consisting of a C₁-C₄ N-alkyl acrylamide, a C₁-C₄ N,N-dialkyl acrylamide, and a combination thereof and (b) an alkyl acrylate selected from the group consisting of a C₅-C₁₈ alkyl acrylate, a C₅-C₁₈ alkyl methacrylate, and a combination thereof, wherein the alkyl acrylate is at most 3 weight percent of the copolymer. The hydrogels can be used to occlude a duct or channel in a subject. For example, the hydrogels can occlude a tear duct, which has numerous medical benefits as described herein.

### CROSS-REFERENCE TO RELATED APPLICATIONS

This application claims the benefit of and priority to co-pending U.S. Provisional Patent Application No. 63/661,185, filed on Jun. 18, 2024, the contents of which are incorporated by reference herein in their entireties.

### BACKGROUND

The occlusion of ducts and channels in a patient has numerous medical applications. For example, punctal plugs are a mechanical device inserted into the tear ducts which conserve tears on the surface of the eye. These plugs are commonly used to improve symptoms of Dry Eye Disease by increasing tear volume and thereby alleviating irritation, discomfort, and other associated conditions. Punctal Plugs are not just used for dry eye disease, however, and have been shown to improve punctal stenosis, support ocular pharmaceutical retention, repair corneal lesions, enhance tear film stability, and improve contrast sensitivity. These uses, among others in ophthalmic practice result from the same property—mechanical occlusion of the tear duct.

Punctal plugs are physician-administered and can last for years when working properly, but they suffer from their own issues. They were invented in 1975 and come in several forms. Pre-molded silicone plugs have been used for decades as a permanent treatment while collagen plugs work on the scale of weeks to a month. There is no superior plug or standard of care. This is due to persistent problems with fit, removal, and comfort. Extrusion, when the plug falls out of the duct, occurs at a rate of 25-50% in the two-month to two-year time frame after insertion. One study observed a 37% extrusion rate within six months of insertion, illustrating the immediacy of the issue, which requires increased time in clinic and cost for replacement. Size mismatch and duct flexibility can also lead to migration. Some problematic plugs with a migration rate of 6.6% have led some physicians to avoid plugs entirely. Many plugs are not designed for removal, leading to patients where multiple plugs have been found in ducts upon surgical removal that were thought to have been removed by irrigation. These problems persist and there has been little to no innovation in this product category for 20 years.

### SUMMARY

Described herein are hydrogels composed of binary copolymers. In one aspect, the copolymer comprises at least one residue from (a) an acrylamide selected from the group consisting of a C₁-C₄ N-alkyl acrylamide, a C₁-C₄ N,N-dialkyl acrylamide, and a combination thereof and (b) an alkyl acrylate selected from the group consisting of a C₅-C₁₈ alkyl acrylate, a C₅-C₁₈ alkyl methacrylate, and a combination thereof, wherein the alkyl acrylate is at most 3 weight percent of the copolymer. The hydrogels can be used to occlude a duct or channel in a subject. For example, the hydrogels can occlude a tear duct, which has numerous medical benefits as described herein.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

Described herein are copolymers and hydrogels prepared from the same that enables a wide range of material properties and behaviors. The copolymer is used to engineer precise behaviors in stimulus responsive polymers such that they that undergo a controlled change in material properties, behavior, or structure upon a selected environmental stimulus. The specific condition at which this change occurs is referred to as the trigger point. These polymers may require formulation with solvents or other excipients to elicit these behaviors.

The copolymers and hydrogels can be adapted to a wide range of applications including but not limited to medical sealing, drug release, cosmetics, skincare, industrial materials, smart apparel, and additive manufacturing.

### Definitions

Unless otherwise stated, the following terms in this application have the definitions given below. The Section headings used herein are for organizational purposes only and are not to be construed as limiting the subject matter described.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

As used herein, a plurality of items, structural elements, compositional elements, and/or materials may be presented in a common list for convenience. However, these lists should be construed as though each member of the list is individually identified as a separate and unique member. Thus, no individual member of any such list should be construed as a de facto equivalent of any other member of the same list based solely on its presentation in a common group, without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range was explicitly recited. As an example, a numerical range of “about 1” to “about 5” should be interpreted to include not only the explicitly recited values of about 1 to about 5, but also to include individual values and sub-ranges within the indicated range. Thus, included in this numerical range are individual values such as 2, 3, and 4, the sub-ranges such as from 1-3, from 2-4, from 3-5, from about 1-about 3, from 1 to about 3, from about 1 to 3, etc., as well as 1, 2, 3, 4, and 5, individually. The same principle applies to ranges reciting only one numerical value as a minimum or maximum. The ranges should be interpreted as including endpoints (e.g., when a range of “from about 1 to 3” is recited, the range includes both of the endpoints 1 and 3 as well as the values in between). Furthermore, such an interpretation should apply regardless of the breadth or range of the characters being described.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc. of these materials are disclosed, that while specific reference to each various individual combination and permutation of these compounds may not be explicitly disclosed, each is specifically contemplated and described herein. For example, if a thermoresponsive polymer is disclosed and discussed, and a number of different additives are discussed, each and every combination of thermoresponsive polymer and additive that is possible is specifically contemplated unless specifically indicated to the contrary. For example, if a class of thermoresponsive polymers A, B, and C are disclosed, as well as a class of additives D, E, and F, and an example combination of A+D is disclosed, then even if each is not individually recited, each is individually and collectively contemplated. Thus, in this example, each of the combinations A+E, A+F, B+D, B+E, B+F, C+D, C+E, and C+F is specifically contemplated and should be considered from disclosure of A, B, and C; D, E, and F; and the example combination A+D. Likewise, any subset or combination of these is also specifically contemplated and disclosed. Thus, for example, the sub-group of A+E, B+F, and C+E is specifically contemplated and should be considered from disclosure of A, B, and C; D, E, and F; and the example combination of A+D. This concept applies to all aspects of the disclosure including, but not limited to, steps in methods of making and using the disclosed compositions. Thus, if there are a variety of additional steps that can be performed with any specific embodiment or combination of embodiments of the disclosed methods, each such composition is specifically contemplated and should be considered disclosed.

In the specification and in the claims that follow, reference will be made to a number of terms that shall be defined to have the following meanings:

It must be noted that, as used in the specification and the appended claims, the singular forms “a,” “an,” and “the” include plural referents unless the context clearly dictates otherwise. Thus, for example, reference to “an additive” includes mixtures of two or more additives and the like.

“Optional” or “optionally” means that the subsequently described event or circumstance can or cannot occur, and that the description includes instances where the event or circumstance occurs and instances where it does not.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

It should be noted that ratios, concentrations, amounts, and other numerical data can be expressed herein in a range format. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. Ranges can be expressed herein as from “about” one particular value and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. For example, an acrylic acid residue in a copolymer used herein refers to one or more —CH₂CH(CO₂H)— units in the copolymer, regardless of whether acrylic was used to produce the copolymer.

As used herein, the term “polymer” may refer to a homo-polymer, a copolymer, a tri-polymer and other multi-polymer, or a mixture thereof.

As used herein, the term “binary copolymer” may refer to a copolymer derived from an acrylamide and an alkyl acrylate as described herein.

As used herein, the term “vinyl polymer” includes all polymers derived from vinyl monomers which have a backbone chain comprised of covalently linked carbon atoms. Vinyl polymers may be homopolymers, copolymers with 2 or more constituent monomer groups, cross-linked, or mixed. Cross linked vinyl polymers may have backbones which are not exclusively covalently bonded carbon atoms, or backbone regions which are not exclusively covalently bonded carbon atoms.

As used herein, the term “lower critical solution temperature” (LCST) or “lower consolute temperature” is the critical temperature below which the copolymer described herein is miscible for all compositions.

The term “upper critical solution temperature” (UCST) or “upper consolute temperature” is the critical temperature above which the copolymer described herein is miscible in all proportions.

The term “gelation point” refers to the condition at which an environmentally responsive formulation undergoes a phase transition to a more gelled state, reflected in a loss of fluidity. Gelation point and cloud point (Tcp) are used interchangeably.

The term “trigger point” refers to the condition upon which an environmentally responsive formulation undergoes a change in material properties, behavior, or structure. In some embodiments, a trigger point may be a gelation point or a LCST.

As used herein, the term “admixing” is defined as mixing two or more components together so that there is no chemical reaction or physical interaction. The term “admixing” also includes the chemical reaction or physical interaction between the two or more components.

As used herein, the term “subject” or “individual” as used herein includes mammals. Non-limiting examples of mammals include humans, dogs, cats, and mice, including transgenic and non-transgenic mice. The methods described herein can be useful in both human therapeutics, pre-clinical, and veterinary applications. In some embodiments, the subject is a mammal, and in some embodiments, the subject is human.

The terms “prevent,” “preventing,” or “prevention” refer to providing treatment prior to the onset of a condition. If treatment is commenced in subjects with a condition, such treatment is expected to prevent, or to prevent the progression of, the medical sequelae of the condition.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “tear duct” or “tear ducts” as used here in refers to any portion of the lacrimal system, such as puncta, canaliculi, lacrimal sac, and nasolacrimal duct.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can include one or multiple documents and are meant to include future updates.

### Hydrogels

Described herein are hydrogels that change their physical properties upon a known stimulus. In one aspect, the hydrogels are activatable between a delivery state and a deployed state. In some instances, the hydrogels can be activatable between a therapeutic state and a release state.

In one aspect, in the delivery state, the hydrogels described herein are in liquid or semi-liquid (e.g., gel) in form. This allows them to be easily delivered into an orifice or channel in a body of an individual. In this embodiment, the deployed state is preferably a solid state. In the release state, the hydrogels are preferably liquid or semi-liquid, which allows the polymers to easily be removed from the duct or channel.

In another aspect, the hydrogels have a liquid or semi-liquid delivery state, and a solid or semi-solid deployed state. The hydrogels may not be a release state or the activatable feature of the hydrogels may only slightly alter the material properties of the polymer hydrogel when exposed to certain stimuli (e.g. alkaline solution, acidic solution, buffer solution, saline irrigation, probing with forceps, aspiration).

Environmental stimuli that may cause a change in the hydrogel include, but are not limited to, temperature, light, pH, sound, presence of a specific excipient, presence of a specific biological molecule or material, mechanical force, electric fields, and magnetic fields.

In certain aspects the copolymer in the hydrogel is a thermoresponsive polymer. Thermoresponsive polymers are polymers that exhibit a change of their physical properties with temperature. Depending upon the thermoresponsive polymer, a drastic change to its physical properties can occur with a change of environmental temperature. In some aspects, a thermoresponsive polymer exhibits a volume phase transition at a certain temperature. In other aspects, a change in supermolecular architecture, color, miscibility, or other material property can be exhibited. This transition can be reversible or irreversible. Reversal to the original condition may also be gated by specific durations or environmental conditions.

In response to such environmental stimuli, the copolymer may undergo one or more changes in physical or chemical properties. In some embodiments this change can affect toughness, cohesiveness, adhesiveness, color, opacity, conductivity, or solubility.

The copolymer can have one or more trigger points at which changes occur. These trigger points may concern the same or a different environmental stimulus. These trigger points may also concern the same or different type of change in material property. For example, the copolymer may undergo an increase in cohesion at one trigger point because of a change in temperature, and an increase in adhesion at another due to a change in local pH.

In certain aspects the copolymer comprises a responsive monomer and one or more weighting monomers which impact the trigger point. These weighting monomers can have raising or lowering effects on the trigger point or amplifying effects that alter the severity or duration of the change in copolymer or formulation properties.

In certain aspects, the copolymer can be crosslinked but only to an extent that their environmentally sensitive properties are not lost. In other aspects, cross-linking is intentionally performed to inhibit environmental sensitivity. In other aspects, multiple copolymers can be a mixture of crosslinked and linear chains that allow for a balancing of properties.

In one embodiment, the copolymer comprises at least one residue from (a) an acrylamide selected from the group consisting of a C₁-C₄ N-alkyl acrylamide, a C₁-C₄ N,N-dialkyl acrylamide, and a combination thereof and (b) an alkyl acrylate selected from the group consisting of a C₅-C₁₈ alkyl acrylate, a C₅-C₁₈ alkyl methacrylate, and a combination thereof, wherein the alkyl acrylate is at most 3 weight percent of the copolymer.

In certain embodiments the group A monomer is selected from n-isopropylacrylamide, N-propylacrylamide, N-propylmethacrylamide, N-ethoxyethylacrylamide, N-tetrahydrofurfurylacrylamide, N-tetrahydrofurfurylmethacrylamide, N,N-diethylacrylamide, N-isopropylmethacrylamide, N-tert-butylacrylamide, N-ethylacrylamide, N-cyclopropylacrylamide, N,N-dimethylacrylamide, N,N-diethylacrylamide, N-ethylacrylamide, or any combination thereof.

In certain aspects, the copolymer includes an additional monomer or group of monomers that decreases solvent affinity and thereby a gelation point (Group B).

In certain aspects, Group B monomers are selected from alkyl acrylates and alkyl methacrylates. In certain aspects, Group B monomers are one or more C₅-C₁₅ alkyl acrylate. Examples of alkyl acrylates include, but are not limited to, pentyl acrylate, hexyl acrylate, 2-ethylhexyl acrylate, heptyl acrylate, octyl acrylate, nonyl acrylate, decyl acrylate, undecyl acrylate, dodecyl acrylate, hexadecyl acrylate, stearyl acrylate, lauryl acrylate, isobornyl acrylate and any combination thereof.

In some aspects, the incorporation of Group B monomers into the copolymer decreases a Tcp in a solvent, resulting in gelation at lower temperatures than if the Group B monomer were excluded from the structure.

In some aspects, the incorporation of a type B monomer improves the durability of the copolymer. As a hydrogel this may be observed as a more cohesive gel state above a Tcp. Not wishing to be bound by theory, one possible mechanism is that the alkyl chains entangle or cluster, causing greater cohesion. Longer chain alkyl groups can further cause swelling in the solvent, resulting in higher viscosity below a Tcp, and a more solid form above Tcp.

In certain aspects, a strengthening monomer is added to the copolymer to improve material properties of the hydrogel. This monomer may be used to change the swelling behavior below a gelation point, improve solubility in a solvent system, improve durability above the gelation point, control release of a bound, entangled, or encapsulated active ingredient, alter the gelation temperature, or imbue the copolymer with an additional environmental sensitivity.

In one aspect, the strengthening monomer is a cross-linker. These monomers have two or more vinyl functional groups that allow the linking of multiple polymer chains. Glycol diacrylates, other diacrylates, and bisacrylamides are examples of some linking monomers used. In some embodiments these monomers are comprised of polyethyleneglycol diacrylate, bisacrylamide and methylene bisacrylamide.

In another aspect, the strengthening monomer is a multifunctional acrylate or methacrylate. In certain embodiments these monomers include 1,1,1-Trimethylolpropane triacrylate, 1,1, 1-trimethylolpropane trimethacrylate, pentaerythritol tetraacrylate, and dipentaerythritol pentaacrylate.

In some aspects, the cross-linker is included up to 1 weight percent of the copolymer. In another aspect, the crosslinker is from about 0.05 weight percent, 0.1 weight percent, 0.25 weight percent, 0.5 weight percent, 0.75 weight percent, or 1.0 weight percent, where any value can be a lower and upper endpoint of a range (e.g., 0.25 weight percent to 0.5 weight percent).

In another aspect, an additional monomer is added to the copolymer that enables storage and controlled release of pharmaceuticals or other excipients. In one aspect, this monomer binds or sequesters an excipient for later release. In one aspect, the monomer includes a binding region such as 1) arylsulfate, disulfide or peptide—cleavable by enzyme, 2) hydrazone or acetyl—cleavable by acid, 3) disulfide—cleavable by glutathione, 4) nitrobenzyl or coumarin—cleavable by light, 5) azide-alkyne or thiolene—cleavable by click chemistry, 6) maleimide—hydrolysis or glutathione or alternate reducing agent.

In certain aspects, the properties of the hydrogel can be tailored by increasing the molecular weight of the copolymer used. In some aspects, a cross-linker may be used to achieve a higher molecular weight.

In certain aspects, prior to administration to the subject, the hydrogel is subjected to irradiation. In one aspect, this irradiation is accomplished by gamma irradiation. In other aspects, it is accomplished by e-beam. In one aspect, the hydrogel is subjected to electron beam irradiation between about 5 kGY and about 45 kGY, 5 kGY, 10 kGY, 15 kGY, 20 kGY, 25 kGY, 30 kGY, 35 kGY, 40 kGY, or 45 kGY, where any value can be a lower and upper endpoint of a range (e.g., 10 kGY to 20 kGY).

In one aspect, the hydrogel is subjected to electron beam radiation in a single dose or in two or more sequential doses. In one aspect, the hydrogel is subjected to electron beam radiation in multiple doses such that the hydrogel is exposed to a total dosage of radiation in the range of about 20 kGY and about 45 kGY. For example, the hydrogel can be subjected to a first dose of electron beam radiation ranging from of about 10 kGY and about 20 kGY, a subsequent second dose of electron beam radiation ranging from of about 10 kGY and about 20 kGY, and a subsequent third dose of electron beam radiation ranging from of about 10 kGY and about 20 kGY, where the total amount of radiation is from about 20 kGY and about 45 kGY.

In one aspect, prior and/or subsequent to exposure to irradiation, the hydrogel is pre-conditioned. In one aspect, the hydrogel is cooled to a temperature of about −20° C. and 10° C. In another aspect, the hydrogel is cooled to a temperature of about −20° C., −15° C., −10° C., −5° C., 0° C., 5° C., or 10° C., where any value can be a lower and upper endpoint of a range (e.g., 0° C. to 10° C.). Not wishing to be bound by theory, pre-cooling of the hydrogel prior to exposure to irradiation reduces the risk of the hydrogel reaching the critical transition range during irradiation. In one aspect, if the hydrogel is exposed to two or more does of electron beam radiation, the hydrogel is pre-cooled prior to the subsequent dose of irradiation. Not wishing to be bound by theory, pre-conditioning the hydrogel prior to subjecting the hydrogel to electron beam radiation prevents undesirable thermal rearrangement.

The copolymer can have a variety of compositions depending on the desired properties. For example, the Group A monomer can impart environmental response. In certain embodiments the Group A monomer is the majority monomer, occupying a higher mol percent of the polymer composition than any other groups respectively. In certain aspects, the Group A monomer is from 50 weight % to 97 weight % of the copolymer. In another aspect, the Group A monomer is 50 weight %, 55 weight %, 60 weight %, 65 weight %, 70 weight %, 75 weight %, 80 weight %, 85 weight %, 90 weight %, 95 weight %, or 97 weight %, where any value can be a lower an upper endpoint of a range (e.g., 75 weight % to 90 weight %)

In one aspect, the residue from Group (b) is from about 0.05 weight percent to up to 3 weight percent of the copolymer. In another aspect, the residue from Group (b) is from about 0.05 weight percent, 0.1 weight percent, 0.5 weight percent, 1.0 weight percent, 1.25 weight percent, 1.5 weight percent, 1.75 weight percent, 2.0 weight percent, 2.25 weight percent, 2.5 weight percent, 2.75 weight percent, or 3.0 weight percent, where any value can be a lower and upper endpoint of a range (e.g., 1.0 weight percent to 2.0 weight percent).

In one aspect, the hydrogel may include two or more different copolymers as described herein, where multiple copolymer and ratios of the copolymers may or may not be held constant. For example, to achieve unique formulation properties, compositional ratios may be retained but copolymers with different average molecular weights incorporated. As another example, two copolymers with entirely different compositional elements may be used to facilitate multiple environmental responses.

The molecular weight of the copolymers described herein can affect or modify the properties of the hydrogels. In one aspect, the copolymer has a number average molecular weight of about 10,000 daltons to about 300,000 daltons. In another aspect, copolymer has a number average molecular weight of about 10,000 daltons, 50,000 daltons, 100,000 daltons, 150,000 daltons, 200,000 daltons, 250,000 daltons, or 300,000 daltons, where any value can be a lower and upper endpoint of a range (e.g., 50,000 daltons to 150,000 daltons).

The properties of the copolymer are designed around one or more trigger points. In certain aspects, a trigger point is from about 5° C. to about 80° C., or about 25° C., about 28° C., about 32° C., about 34° C., about 36° C., about 37° C., about 40° C., about 50° C., about 60° C., about 70° C., about 80° C., where any value can be a lower and upper endpoint of a range (e.g., about 18° C. to about 32° C., about 25° C. to about 36° C., etc.). In certain aspects, the trigger point is centered around physiological body temperature.

In one aspect, the copolymer the copolymer has a lower critical solution temperature from about 20° C. to about 35° C. in aqueous solution, or 20° C., 21° C., 22° C., 23° C., 24° C., 25° C., 26° C., 27° C., 28° C., 29° C., 30° C., 31° C., 32° C., 33° C., 34° C., or 35° C., where any value can be a lower and upper endpoint of a range (e.g., about 25° C. to about 32° C.).

The balancing effects of copolymers on the trigger point of the hydrogels can be modified. As demonstrated in the Examples, it can be observed that as the relative portion of alkyl acrylate in the copolymer increases, the Tcp trigger point lowers.

The hydrogels described herein may be used for a variety of medical or consumer applications. To achieve certain applications, combinations of the copolymers with various solvents and excipients can be employed.

The copolymers herein can be formulated in an aqueous solution to produce hydrogels. In one aspect, the concentration of the copolymer is from 10 weight percent to about 90 weight percent of the hydrogel. In another aspect, the copolymer can be formulated in an aqueous solution at a concentration of no more than 80%, 75%, 70%, 65%, 60%, 55% or 50%. In another aspect, the concentration of the copolymer is 10 weight percent, 15 weight percent, 20 weight percent, 25 weight percent, 30 weight percent, 35 weight percent, 40 weight percent, 45 weight percent, 50 weight percent, 55 weight percent, or 60 weight percent, where any value can be a lower and upper endpoint of a range (e.g., 20 weight percent to 40 weight percent).

In certain aspects, the hydrogels benefit from incorporation of one or more secondary polymers. Secondary polymers can provide additional material properties or behaviors such as, for example, strength, flexibility, gloss, hardness, differences in environmental response, or impacts on formulation characteristics like solubility.

In certain aspects the secondary polymer is a cellulose ester, wherein the cellulose ester comprises cellulose acetate butyrate, nitrocellulose, cellulose acetate propionate, cellulose acetate phthalate, hydroxypropyl cellulose, hydroxyethyl cellulose, or any combination thereof. In certain aspects the polymer is an alkylated cellulose such as methyl cellulose or ethyl cellulose. Each of these cellulose forms can be purchased or manufactured in a range of molecular weights and constituent ratios.

In one aspect, the secondary polymer is a polyurethane, an acrylic resin, a cellulose ester, a rosinate, or any combination thereof.

In certain embodiments, the secondary polymer is capable of forming a hydrogel or dissolving in water and is selected from polyethylene glycol, polyvinyl alcohol, polyacrylamide, alginate, chitosan, hyaluronic acid, gelatin, polyvinyl pyrrolidone, polyglycolic acid, polylactic acid, polydioxanone, caprolactone, polyvinyl caprolactone, polyglyconate, collagen, and fibrin.

In certain other aspects, the secondary polymer is a vinyl polymer formed from acrylic monomers.

In one aspect, the secondary polymer is from about 0.2 weight percent to about 20 weight percent of the hydrogel. In another aspect, the secondary polymer is about 0.2 weight percent, about 5% about 10 weight percent, about 15 weight percent, or about 20 weight percent of the hydrogel, where any value can be a lower and upper endpoint of a range (e.g., 6 weight percent to 12 weight percent). In other embodiments the secondary polymer is 30-55 weight percent of the hydrogel.

In certain aspects, the hydrogels described herein include one or more additives. The additives can modify physical properties, chemical properties, or health benefits of the formulations. In one aspect, when the hydrogel is for topical use, the additive can include a polymer, ceramics, metals, salt, a rheology modifier, a suspension agent, a dispersant, a wetting agent, a low vapor pressure solvent, a plasticizer, pigments, effect powders, fragrance, an antimicrobial agent, an emollient, an emulsifier, a solubilizer, a humectant, a stabilizer, an exfoliant, a lipid or oil, a vitamin, a biological molecule, an essential oil, a fragrance, a drug carrier, a preservative, a thickener, color additive, pH adjuster, a surfactant, a pharmaceutical, an adhesion promoter, or any combination thereof.

In certain aspects ceramic materials are incorporated into the hydrogels. In some embodiments these material are selected from alumina, zirconia, hydroxyapatite, kaolin, bentonite, silica, feldspar, sand, or a combination thereof.

When the hydrogel needs a desired color or appearance, one or more pigments are incorporated into the hydrogel. Examples of pigments include, but are not limited to, 3158TPLitholRubine BK, Carbon black N330, Degussa Printex, Titanium dioxide, D&C Brown No 1, D&C Black No 2, D&C Black No 3, 1104 Fast Yellow, 1283 Permanent Yellow HR, 1646 Medium Chrome yellow, FD&C Yellow No 5, D&C Yellow No 5 lake, D&C Yellow No 6 lake, D&C Yellow No 7 lake, D&C Yellow No 8, D&C Yellow No 10, D&C Yellow No 11, 3157 Vulcan Red LC, FD&C Red 3 lake, FD&C Red 4 lake, D&C Red No 4 aluminium lake, D&C Red No 6 aluminium lake, D&C Red No 6 barium lake, D&C Red No 7 lake, D&C Red No 8 lake, D&C Red No 9 lake, D&C Red No 17, D&C Red No 21, D&C Red No 22, D&C Red No 27 lake, D&C Red No 28, D&C red No 30 lake, D&C Red No 31, D&C Red No 33 lake, D&C red No 34 lake, D&C Red No 36 lake, FD&C Red No 40, 4421 Phthalocyanine Blue B pigment, 4431 Phthalocyanine Blue BS pigment, 4311 Pigment Sky Blue B, FD&C Blue 1 lake, D&C Blue No 4, 5319 Fast Green lake pigment, 5406 Phthalocyanine Green G pigment, 3327 Basic Rhodamine BG Lake pigment, FD&C Green No 3, D&C Green No 3 lake, D&C Green No 5, D&C Green No 6, D&C Green No 8, 6360 Fast Violet lake pigment, D&C Violet No 2, D&C Orange No 4 lake, D&C Orange No 5 aluminium lake, D&C Orange No 5 zirconium lake, D&C Orange No 10 aluminium lake, D&C Orange No 11, D&C Orange No 17 lake, Iron oxides of various shades, manganese violet, silver nitrate, ultramarines, zinc oxide, ferric ammonium ferrocyanide, ferric ferrocyanide, aluminum powder, bismuth oxychloride, copper powder, chromium hydroxide green, chromium oxide green, silver, Mica, or a combination thereof.

In one aspect, the pigment is from about 0.5 weight percent about 10% of the total weight of the hydrogel. In another aspect, the pigment is from about 0.5 weight percent, about 1 weight percent, about 2 weight percent, about 3 weight percent, about 4 weight percent, about 5 weight percent, about 6 weight percent, about 7 weight percent, about 8 weight percent, about 9 weight percent, or about 10 weight percent of the hydrogel, where any value can be a lower and upper endpoint of a range (e.g., 3 weight percent to 7 weight percent).

In one aspect, rheology modifiers can be used to alter formulation material properties. In one aspect, a filler such as, for example, hectorite, stearalkonium hectorite, bentonite, bentonite, stearalkonium bentonite, silica, hydrophobic fumed silica, hydrophilic fumed silica, silica dimethyl silylate, organophilic phyllosilicate, kaolin clay, nanoclay, halloysite, montmorillonite, or any combination thereof can be used.

In one aspect, the filler is from about 0.5 weight percent about 10% of the total weight of the hydrogel. In another aspect, the filler is from about 0.5 weight percent, about 1 weight percent, about 2 weight percent, about 3 weight percent, about 4 weight percent, about 5 weight percent, about 6 weight percent, about 7 weight percent, about 8 weight percent, about 9 weight percent, or about 10 weight percent of the hydrogel, where any value can be a lower and upper endpoint of a range (e.g., 3 weight percent to 7 weight percent).

In other aspects, a high amount of filler is used at weight percents of 30-60% by mass of the total formulation composition.

In certain aspects, dispersants and wetting agents are used to improve the uniformity of formulations and prevent aggregation. Examples include, but not limited to, glycols, surfactants, ethoxylated alcohols, polymeric wetting agents like ethylene oxide-propylene oxide block copolymers, and silicones. In some embodiments the wetting agent in the formulations is styrene/acrylates copolymer, acrylic block copolymer, ethylene oxide/propylene oxide block copolymer, stearic acid, jojoba esters, glycerol, propylene glycol, ethylene glycol, lactonic sophorolipid, cocoamido propyl dimethyl amine, alpha-arbutin, cocamidopropylamine oxide, 2-octyldodecyl 2-ethylhexanoate, 1, 2, 3-trilinolenoylglycerol, cyclopentanecarboxylic acid sodium lauryl sulfate, octyl phenol ethoxylate, polysorbate 20, dimethicone, amodimethicone, or any combination thereof.

In certain aspects, plasticizers are incorporated to improve the flexibility, smoothness, lubricity, or crack resistance of the formulation. Examples include, but not limited to, acetyl tributyl citrate, tributyl citrate, triethyl citrate, dimethyl adipate, glycerin propylene glycol, ethylene glycol monoricinoleate, 2,2,4-trimethyl-1,3-pentanediol monoisobutyrate, bis(2-ethylhexyl) sebacate, diisopropyl sebacate, tridodecyl phosphate, dimethyl adipate, lavender essential oil, jojoba oil, argan oil, essential oils, plant oils, or a combination thereof. Some surfactants, resins, and other ingredients listed elsewhere in this disclosure with low vapor pressure are also suitable as plasticizers.

In one aspect, an additive is an anti-microbial agent including, but not limited to, tetrasodium EDTA, disodium EDTA, phenoxyethanol, diazolidinyl urea, iodopropynyl butylcarbamate, Lactobacillus & Cocos Nucifera (coconut) fruit extract, Leuconostoc ferment filtrate, benzyl alcohol, salicylic acid, glycerin, sorbic acid, gluconolactone, Sorbus aucuparia fruit ferment filtrate, capryl glycol, Sambucus nigra fruit extract, or any combination thereof.

In one aspect, the additive is an emollient including, but not limited to, shea butter, cocoa butter, mineral oil, lanolin, petrolatum, paraffin, beeswax, squalene, coconut, jojoba, sesame, avocado, argan, jojoba, almond, castor, and other plant oils (hydrogenated or unhydrogenated), cetyl alcohol, olive oil (e.g., oleic acid), triethylhexanoin, copernicia cerifera wax, lauryl laurate, capric triglyceride, cetearyl alcohol, alkyl benzoate, glyceryl stearate, coceth-20, glycerin, hydroxyetheyl cellulose, potassium sorbate, sodium phytate, citric acid, Lactobacillus ferment, behenyl alcohol, Nereocystis luetkeana ferment filtrate, hydrogenated polyisobutene, or any combination thereof.

In one aspect, the additive is an emulsifier including, but not limited to, cetearyl alcohol, glyceryl stearate, sodium stearoyl lactylate, PEG-100 stearate, behentrimonium methosulfate, polysorbate, sodium acrylate, isohexadecane, acacia gum, PEG, PEG-7 glyceryl monococoate, PEG-40 hydrogenated castor oil, PEG distearate, sorbitan laurate, polyglyceryl laurate, dilauryl citrate, caprylic triglyceride, cetearyth-n, sodium polyacrylate, ehthylhexyl cocoate, PPG-3 Benzyl ether myristate, or any combination thereof.

In one aspect, the additive is a solubilizer including, but not limited to, capryl glucoside, ethoxydiglycol, sodium cocoyl glutamate, glyceryl caprylate, polyglyceryl-6 oleate, sodium surfactin, D-glucopyranose, decyl octyl glycosides, polyglyceryl caprylate, polyglyceryl caprate, polyglyceryl cocoate, polyglyceryl ricinoleate, or any combination thereof.

In one aspect, the additive is a humectant including, but not limited to, propanediol, butylene glycol, ethoxy diglycol, ethylhexyl glycerin, pentylene glycol, propylene glycol, silica gel, PPG-20 methyl glucose ether, glycerin, malic acid, honey, honey powder, or any combination thereof.

In one aspect, the additive is a stabilizer including, but not limited to, xanthum gum, glucose oxidase, sclerotium gum, behenyl alcohol, cetyl alcohol, candellia wax, arrowroot powder, plant wax, sorbitan olivate, or any combination thereof.

In one aspect, the additive is an exfoliant including, but not limited to, salicylic acid, citric acid, glycolic acid, urea, plant powders, plant acids, glucosamine, plant extracts, enzymatic products of bacterial fermentation, or any combination thereof.

In one aspect, the additive is a binder selected from acrylic polymers, alkyds, polyurethanes, vinyl acetate, epoxy, polyvinyl chloride, latex, chitosan, casein, rubber acrylic resins, or combinations thereof.

In one aspect, the additive is a lipid or oil including, but not limited to, ceramides, blueberry seed oil, acacia seed oil, algae oil, almond oil, black currant extract, blackberry seed oil, borrage oil, jasmine oil, pomegranate seed oil, raspberry seed oil, other plants oils and extracts, cholesterol, phospholipids, linoleic acid, or combinations thereof.

In one aspect, the additive is a peptide, pre-peptide, or other biological molecule including, but not limited to, collagen pre-peptide, collagen, palmitoyl pentapeptide-4, palmitoyl oligopeptide, palmitoyl tetrapeptide-7, ferment peptide extracts, or plant extract derived peptides.

In one aspect, the additive is a surfactant. In another aspect, the surfactant is a nonionic surfactant. Examples of nonionic surfactants include the condensation products of a higher aliphatic alcohol, such as a fatty alcohol, containing about 8 to about 20 carbon atoms, in a straight or branched chain configuration, condensed with about 3 to about 100 moles, preferably about 5 to about 40 moles, most preferably about 5 to about 20 moles of ethylene oxide. Examples of such nonionic ethoxylated fatty alcohol surfactants are the Tergitol™ 15-S series from Union Carbide and Brij™ surfactants from ICI. Tergitol™ 15-S Surfactants include C11-C15 secondary alcohol polyethyleneglycol ethers. Brij™97 surfactant is polyoxyethylene(10) oleyl ether; Brij™58 surfactant is polyoxyethylene(20) cetyl ether; and Brij™ 76 surfactant is polyoxyethylene(10) stearyl ether.

Another useful class of nonionic surfactants include the polyethylene oxide condensates of one mole of alkyl phenol containing from about 6 to 12 carbon atoms in a straight or branched chain configuration, with ethylene oxide. Examples of nonreactive nonionic surfactants are the Igepal™ CO and CA series from Rhone-Poulenc. Igepal™CO surfactants include nonylphenoxy poly(ethyleneoxy)ethanols. Igepal™ CA surfactants include octylphenoxy poly(ethyleneoxy)ethanols.

Another useful class of hydrocarbon nonionic surfactants include block copolymers of ethylene oxide and propylene oxide or butylene oxide. Examples of such nonionic block copolymer surfactants are the Pluronic™ and Tetronic™ series of surfactants from BASF. Pluronic™ surfactants include ethylene oxide-propylene oxide block copolymers. Tetronic™ surfactants include ethylene oxide-propylene oxide block copolymers.

In other aspects, the nonionic surfactants include sorbitan fatty acid esters, polyoxyethylene sorbitan fatty acid esters and polyoxyethylene stearates. Examples of such fatty acid ester nonionic surfactants are the Span™, Tween™, and Myj™ surfactants from ICI. Span™ surfactants include C12-018 sorbitan monoesters. Tween™ surfactants include poly(ethylene oxide) C12-C18 sorbitan monoesters. Myj™ surfactants include poly(ethylene oxide) stearates.

In one aspect, the nonionic surfactant can include polyoxyethylene alkyl ethers, polyoxyethylene alkyl-phenyl ethers, polyoxyethylene acyl esters, sorbitan fatty acid esters, polyoxyethylene alkylamines, polyoxyethylene alkylamides, polyoxyethylene lauryl ether, polyoxyethylene cetyl ether, polyoxyethylene stearyl ether, polyoxyethylene oleyl ether, polyoxyethylene octylphenyl ether, polyoxyethylene nonylphenyl ether, polyethylene glycol laurate, polyethylene glycol stearate, polyethylene glycol distearate, polyethylene glycol oleate, oxyethylene-oxypropylene block copolymer, sorbitan laurate, sorbitan stearate, sorbitan distearate, sorbitan oleate, sorbitan sesquioleate, sorbitan trioleate, polyoxyethylene sorbitan laurate, polyoxyethylene sorbitan stearate, polyoxyethylene sorbitan oleate, polyoxyethylene laurylamine, polyoxyethylene laurylamide, laurylamine acetate, hard beef tallow propylenediamine dioleate, ethoxylated tetramethyldecynediol, fluoroaliphatic polymeric ester, polyether-polysiloxane copolymer, and the like.

In one aspect, a pH adjuster is included in the hydrogels described herein. Examples include, but are not limited to, citric acid, sodium bicarbonate, alkyl siliconates, magnesium hydroxide, triethanolamine, ammonia, aminomethyl-propanol, sugar-amines, protonated sugar amines, or combinations thereof.

In one aspect a color protectant is included in the hydrogels described herein. Examples include, but are not limited to, hydroxyphenyl benzotriazoles, butanedioic acid dimethylester polymer with 4-hydroxy-2,2,6,6-tetramethyl-1-piperidine ethanol, other oligomeric UV stabilizers, bisoctrizole, bemotrizinol, dimethyl (p-methoxybenzylidene)malonate, dioxybenzone, hindered amines, high molecular weight oligomeric hindered amines, assorted antioxidants, hydroxyacetophenone, butylated hydroxytoluene, butylated hydroxyanisole, vitamin E, other vitamins, plant extracts, green tea extract, rosemary extract, resveratrol, UV absorbers, benzophenone, ethylhexyl methoxycinnamate, ethylhexyl salicylate, avobenzone, homosalate, octocrylene, and combinations thereof.

In another aspect, the hydrogels described herein as well as other formulations (e.g., base formulation, protective formulation) discussed in greater detail below can include additional ingredients to improve the scent of the formulations. These include synthetic and natural fragrances. In some embodiments they include essential oils, agrumen, amber, ambergris, ambrette, amyris, benzoin, bergamot, calone, cashmeran, castoreum, citron, civet, clary sage, coumarin, frangipani, frankincense, galbanum, guaiac wood, hedione, heliotrope, iso e super, jasmine, jasmine sambac, labdanum, mimoa, monoi, muguet, musk, myrrh, narcissus, neroli, oakmoss, opopanax, orris, oud, ozone, patchouli, rose, rose de mai, sandalwood, tonka bean, tuberose, anilla, vetiver, ylang ylang, cedarwood, cardamom, lavender, tea tree, lemongrass, eucalyptus, hydrosol, plant water, or combination thereof.

In certain aspects, any of the hydrogels described herein can include synthetic fragrances, ethylvanillin, heliotropine, ionones, aldehydes, calone, ambrox, ethyl maltol, myristic acid, 2-furaldehyde, delta decalactone, delta dodecalactone, 2 methyl 2 pentenoic acid, 2,4 decadienal, 5 methyl 2 phenyl 2 hexenal, acetaldehyde, acetyl propionyl, anisyl, acpric acid, capoic acid, caprylic acid, cyclotene, methyl 3 nonenoate, ethyl 3 hydroxybutyrate, ethyl maltol, lactic acid, lauric acid, pulegone mercaptan, 4-(p-hydroxyphenyl)-2-butanone, 4-hydroxy-2,5-dimethyl-3(2H)-furanone, triethyl citrate, furanone, acetoin, 6-methyl coumarin, 4,5-dimethyl-3-hydroxy-2,5-dihydrofuran-2-one, 2,5 dimethyl 4-methoxy-3(2h)-furanone, 2-methoxy-4-methylphenol, 5-ethyl-3-hydroxy-4-methyl-2(5h)furanone, caproic acid methyl ester, oleic acid ethyl ester, methyl salicylate, 4-hydroxy pentanoic acid y lactone, 5-ethyl-3-hydroxy-4-methyl-2(5h)furanone, 1,5,5,9-tetramethyl-13-oxatricyclo(8.3.0.0)tridecane, isobutyric acid maltol ester, diethyl succinate, phenyl ethyl isothiocyanatem, 4 hydroxy-5-methyl-3-(2h) furanone, 2-ethyl-5-methyl-4-hydroxy-3(2H)-furanone, dihydro coumarin, ethyl 3-hydroxy caproate, furanone acetate, glyceryl triacetate, 1-octen-3-ol, 3-hydroxy-2-butanone, popcorn pyrimidine, 4-hydroxy-2,5-dimethyl-3(2H)-furanone, 5-methyl-2-hepten-4-one, 4-hydroxy-3methyl octanoic acid lactone, 2-buten-1-one, 1-(2,6,6 trimethyl-1,3cyclohexadien-1-yl, 3-cyclohexene-1carboxaldehyde, 2,4-dimethyl, butyl butyryl lactate, 1,2-di-[(1-ethoxy) ethoxy]propane, 4-(2,6,6-trimethyl cyclo-1-enyl)but-2-en-4-one, butanedioic acid, terpineol, 3-methyl-5-cyclopentadecen-1-one, 5-methyl-2-propan-2-ylhex-2-enal, related aromatic compounds, or any combination thereof.

In certain aspect, oils, vitamins, peptides, proteins, and other ingredients beneficial for health are incorporated into the formulations. In some embodiments these include alpha hydroxy acids, hyaluronic acid, retinol, salicylic acid, ceramides, sulfur, vitamin A, vitamin e, vitamin c, vitamin b, vitamin k, vitamin D, alpha-linolenic acid (ALA), eicosapentaenoic acid (EPA), and docosahexaenoic acid (DHA), calcium carbonate, calcium citrate, keratin, collagen, olive oil, rapeseed oil, jojoba oil, argan oil, essential oils, plant extracts, zinc gluconate, iron (II) sulfate, magnesium oxide, Phytosphingosine, tocopherol acetate, ashwagandha, curcumin, amino acids, peptides, piperine, chavicine, keratin, horsetail extract, amla extract, chamomile extract, sea buckthorn oil, sea buckthorn extract, centella asiatic extract, pin bark extract, plum extract, coffeeberry extract, and combinations thereof.

In certain aspects, the additive is a polymer including, but not limited to, hyaluronic acid, alginate, ethyl cellulose, polyvinyl alcohol, chitosan, poly-ethylene glycol, polyacrylic acid, dextran, hydroxyethylmethacrylate, lecithin, polysorbate, or combinations thereof.

In certain aspects, the additive is a salt including, but not limited to, NaCl, KCl, NaI, NaBr, Calcium Hydroxide, Calcium Carbonate, Sodium Bicarbonate, or any combination thereof.

In certain aspects, the additive is an anti-microbial agent including, but not limited to, tetrasodium EDTA, disodium EDTA, phenoxyethanol, diazolidinyl urea, iodopropynyl butylcarbamate, Lactobacillus & Cocos Nucifera (coconut) fruit extract, Leuconostoc ferment filtrate, benzyl alcohol, salicylic acid, glycerin, sorbic acid, gluconolactone, Sorbus aucuparia fruit ferment filtrate, capryl glycol, Sambucus nigra fruit extract, or any combination thereof.

In certain aspects, the additive is a mineral or clay including, but not limited to, kaolin clay, bentonite clay, montmorillonite, or any combination thereof.

In certain aspects, the additive is an emollient including, but not limited to, shea butter, cocoa butter, mineral oil, lanolin, petrolatum, paraffin, beeswax, squalene, coconut, jojoba, sesame, avocado, argan, jojoba, almond, castor, and other plant oils (hydrogenated or unhydrogenated), cetyl alcohol, olive oil (e.g., oleic acid), triethylhexanoin, copernicia cerifera wax, lauryl laurate, capric triglyceride, cetearyl alcohol, alkyl benzoate, glyceryl stearate, coceth-20, glycerin, hydroxyetheyl cellulose, potassium sorbate, sodium phytate, citric acid, Lactobacillus ferment, behenyl alcohol, Nereocystis luetkeana ferment filtrate, hydrogenated polyisobutene, or any combination thereof.

In certain aspects, the additive is an emulsifier including, but not limited to, cetearyl alcohol, glyceryl stearate, sodium stearoyl lactylate, PEG-100 stearate, behentrimonium methosulfate, polysorbate, sodium acrylate, isohexadecane, acacia gum, PEG, PEG-7 glyceryl monococoate, PEG-40 hydrogenated castor oil, PEG distearate, sorbitan laurate, polyglyceryl laurate, dilauryl citrate, caprylic triglyceride, cetearyth-n, sodium polyacrylate, ehthylhexyl cocoate, PPG-3 Benzyl ether myristate, or any combination thereof.

In certain aspects, the additive is a solubilizer including, but not limited to, capryl glucoside, ethoxydiglycol, sodium cocoyl glutamate, glyceryl caprylate, polyglyceryl-6 oleate, sodium surfactin, D-glucopyranose, decyl octyl glycosides, polyglyceryl caprylate, polyglyceryl caprate, polyglyceryl cocoate, polyglyceryl ricinoleate, or any combination thereof.

In certain aspects, the additive is an essential oil, hydrosol, customized fragrance molecule, mixed fragrance, or any combination thereof.

In certain aspects, the additive is a thickener, a color additive, a pH adjuster, or any combinations thereof.

In certain aspects, the additive is a preservative including, but not limited to, trisodium edetate, tetrasodium edetate, tocopherol, butyl paraben, propyl paraben, ethyl paraben, methyl paraben, DMDM hydantoin, methylisothiazolinone, phenoxyethanol, quaternium-15, phenoxyethanol, benzoic acid, Sorbic acid, glycols, or any combination thereof.

In aspect, the additive is a surfactant. In another aspect, the surfactant is a nonionic surfactant. Examples of nonionic surfactants include the condensation products of a higher aliphatic alcohol, such as a fatty alcohol, containing about 8 to about 20 carbon atoms, in a straight or branched chain configuration, condensed with about 3 to about 100 moles, preferably about 5 to about 40 moles, most preferably about 5 to about 20 moles of ethylene oxide. Examples of such nonionic ethoxylated fatty alcohol surfactants are the Tergitol™ 15-S series from Union Carbide and Brij™ surfactants from ICI. Tergitol™ 15-S Surfactants include C₁₁-C₁₅ secondary alcohol polyethyleneglycol ethers. Brij™97 surfactant is polyoxyethylene(10) oleyl ether; Brij™58 surfactant is polyoxyethylene(20) cetyl ether; and Brij™ 76 surfactant is polyoxyethylene(10) stearyl ether.

Another useful class of nonionic surfactants include the polyethylene oxide condensates of one mole of alkyl phenol containing from about 6 to 12 carbon atoms in a straight or branched chain configuration, with ethylene oxide. Examples of nonreactive nonionic surfactants are the Igepal™ CO and CA series from Rhone-Poulenc. Igepal™CO surfactants include nonylphenoxy poly(ethyleneoxy)ethanols. Igepal™ CA surfactants include octylphenoxy poly(ethyleneoxy)ethanols.

Another useful class of hydrocarbon nonionic surfactants include block copolymers of ethylene oxide and propylene oxide or butylene oxide. Examples of such nonionic block copolymer surfactants are the Pluronic™ and Tetronic™ series of surfactants from BASF. Pluronic™ surfactants include ethylene oxide-propylene oxide block copolymers. Tetronic™ surfactants include ethylene oxide-propylene oxide block copolymers.

In other aspects, the nonionic surfactants include sorbitan fatty acid esters, polyoxyethylene sorbitan fatty acid esters and polyoxyethylene stearates. Examples of such fatty acid ester nonionic surfactants are the Span™, Tween™, and Myj™ surfactants from ICI. Span™ surfactants include C₁₂-C₁₈ sorbitan monoesters. Tween™ surfactants include poly(ethylene oxide) C₁₂-C₁₈ sorbitan monoesters. Myj™ surfactants include poly(ethylene oxide) stearates.

In certain aspects, the nonionic surfactant can include polyoxyethylene alkyl ethers, polyoxyethylene alkyl-phenyl ethers, polyoxyethylene acyl esters, sorbitan fatty acid esters, polyoxyethylene alkylamines, polyoxyethylene alkylamides, polyoxyethylene lauryl ether, polyoxyethylene cetyl ether, polyoxyethylene stearyl ether, polyoxyethylene oleyl ether, polyoxyethylene octylphenyl ether, polyoxyethylene nonylphenyl ether, polyethylene glycol laurate, polyethylene glycol stearate, polyethylene glycol distearate, polyethylene glycol oleate, oxyethylene-oxypropylene block copolymer, sorbitan laurate, sorbitan stearate, sorbitan distearate, sorbitan oleate, sorbitan sesquioleate, sorbitan trioleate, polyoxyethylene sorbitan laurate, polyoxyethylene sorbitan stearate, polyoxyethylene sorbitan oleate, polyoxyethylene laurylamine, polyoxyethylene laurylamide, laurylamine acetate, hard beef tallow propylenediamine dioleate, ethoxylated tetramethyldecynediol, fluoroaliphatic polymeric ester, polyether-polysiloxane copolymer, and the like.

In certain aspects, the additive is a combination of sodium hyaluronate, niacinamide, ceramide NP, ceramide AP, ceramide EOP, phytosphingosine, cholesterol, sodium lauroyl lactylate, carbomer, xanthan gum, tetrasodium EDTA, essential oils, plant hydrosol, and fragrance.

In certain aspects, the additive is a pharmaceutical. In some embodiments these are analgesics selected from aspirin, ibuprofen, naproxen, acetaminophen, opioids, lidocaine, or capsaicin. In some embodiments these are relaxants selected from cyclobenzaprine, baclofen tizanidine, methocarbamol, carisoprodol, and diazepam. In some embodiments, these are steroids selected from dexamethasone, triamcinolone acetonide, hydrocortisone, prednisone, methylprednisolone, fluticasone, betamethasone, beclomethasone, and clobetasol. In certain embodiments these are antibiotics selected from amoxicillin, azithromycin, doxycycline, ciprofloxacin, or cephalexin. In certain embodiments these are antifungals such as fluconazole, ketoconazole, terbinafine, clotrimazole, or nystatin. In certain embodiments these are anti-clotting agents selected from heparin, enoxaparin, apixaban, aspirin, warfarin, or apixaban. In certain embodiments these are ingredients that promote tissue repair such as epidermal growth factors, hormones, peptides, or polysaccharides. In some embodiments these are immunomodulators such as calcineurin inhibitors, TNF inhibitors, interleukin-6 inhibitors, azathioprine, mycophenolate, mofetil, methotrexate, interferons, or janus kinase inhibitors.

In certain aspects the additive is a pharmaceutical that acts as an anti-depressant, relaxant, anti-inflammatory, anti-biotic, anti-fungal, anti-viral, pain reliever, retinoid, steroid, immunomodulator, acne medication, or scar removal medication.

In certain aspects the additive is an adhesion promoter such as trialkoxysilanes, isocyanates, cyclic ethers like epoxides, peptides like arginine, glycine-aspartic acid, collagen or collagen peptides, adhesive proteins like mussel foot protein-5 or synthetic derivatives, 3,4-dihydroxyphenylanine (DOPA), or polymers with high quantities of amine, carboxyl, hydroxyl, and sulfhydryl groups.

The amount of the additive present in the hydrogel can vary. In certain aspects, the additive is present in a total amount from about 0.1% to about 70% by weight based upon a total weight of the composition. In another aspect, the additive is present in a total amount from about 0.1%, about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, or about 70% by weight based upon a total weight of the composition, where any value can be a lower and upper endpoint of a range (e.g., about 1% to about 5%, about 20% to about 40%, etc.).

The hydrogels are produced by admixing the copolymers described herein with optional additives and other components in water. In one aspect, all components are mixed for a sufficient time such that all components are dissolved or substantially dissolved in water. In some aspects, homogenizers or shearing mixers can be used for mixing. In other aspects, a curing step (i.e., crosslinking) is performed. In certain aspects, this step is performed with UV while in other aspects it is performed using irradiation or heat.

### Applications of Hydrogels

The hydrogels described herein have numerous applications such as, for example, medical and cosmetic applications.

In one aspect, the present disclosure relates to pharmaceutical compositions comprising a therapeutically effective amount of the hydrogel as described herein. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

In one embodiment, pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In one embodiment, the hydrogel can be used to deliver one or more pharmaceutical compounds or therapeutic compounds. In one embodiment, the pharmaceutical compound or therapeutic compound is mixed with the copolymers described herein alone or with water to produce the hydrogel. In another embodiment, the copolymer further includes one or more residues comprising a cleavable group, where the pharmaceutical compound or therapeutic compound is covalently bonded to the cleavable group. In one embodiment, wherein the cleavable group is selected from the group consisting of an arylsulfate, a disulfide, a peptide bond, a hydrazone, an acetyl group, a nitrobenzyl group, coumarin, an azide-alkyne, a thiolene group, or a maleimide.

The hydrogel can be administered to subject in a number of different ways depending upon the disorder to be treated. When treating a skin disorder, the hydrogel can be formulation as a topical formulation as described herein. When addressing an internal disorder, the hydrogel can be formulated as an injectable that can be directly applied to the site of interest.

In one embodiment, the hydrogel can be used to treat one or more skin disorders. For example, the hydrogel can lessen the appearance of a scar, treat inflammation, treats an infection, treat acne, lessen the appearance of wrinkles, provide a barrier for a cut, abrasion, or wound, or promote healing of the skin. In another aspect, the hydrogel can be incorporated into hair products (e.g., shampoos, gels, pastes, etc.) to enhance or improve properties of hair.

In one aspect, the hydrogel can be used to seal a duct or perforation in a subject. The perforation can be an external perforation (e.g., on the skin) or an internal perforation. In one aspect, the hydrogel can be used to occlude or partially block an orifice, for example for in vivo therapeutic uses. Such orifice can be a channel. Such channel can be a tear duct. Other examples of channels that can be occluded or partially blocked include a fallopian tube, a vas deferens, an artery, a blood vessel, and a lumen of a bone. The compositions herein can be used to occlude the orifice or channel thus prohibiting the transfer of liquid or solid compositions in the body. Any of the compositions described herein can be used to treat or prevent dry eye in a subject in need thereof by occluding at least one tear duct. Any of the activatable polymers herein can be used in hydrogels to occlude or partially block a tear duct in a mammal.

In another embodiment, the hydrogel can be used to treat an eye disorder in a subject. For example, the hydrogel can treat dry eye disease, ocular inflammation, contact lens discomfort, glaucoma, or insufficient tear volume. In one aspect, the hydrogel can be administered to a tear duct of a subject in liquid or semi-liquid form. Upon contact with the subject's tear duct, or in response to the subject's body temperature, the hydrogel is activated which results in a phase change converting the copolymer into solid form.

When occluding an orifice such as, for example, a tear duct, subsequent to solidification, the polymer gel herein forms a plug. The plug may be visible using the naked eye. The plug may be visible using a microscope, magnifying glass, or a magnifying instrument. In certain embodiments, the plug may not be visible.

The plug may be removed by mechanical means or by application of a stimulus. Mechanical means include the use of forceps, aspiration, irrigation, application of pressure to the duct, or an applicator tip. For example, mechanical removal of the plug may include locating the plug, inserting the forceps into the channel, engaging the plug, removing the plug, and checking for patency of the channel. Stimuli removal involves applying a stimulus to the plug, such as irrigation, or application of a cold pack, thereby causing the plug to dissolve. In some embodiments the stimulus does not dissolve the gel plug but changes its mechanical properties. In some embodiments, the irrigation fluid is approximately less than the transition temperature. In some embodiments, the irrigation fluid is less than 32° C. The irrigation fluid may be water, saline solution, mineral oil, and the like. In some embodiments, any of the triggers described herein can be used to dissolve, soften, or de-anchor a plug. For example, stimuli removal may include locating the plug, flushing the channel with fluid, and checking the channel for patency of the channel.

### Kits

Described herein are kits that include the hydrogel in a storage device and instructions for use. In certain aspects, the storage device can be fitted with a pump for delivering the topical composition to skin. In other aspects, the kit can include an applicator such as, for example, a dropper, a brush, or an injector to apply the topical composition to the skin surface. The storage device can be made of any material including, but not limited to, glass or plastic.

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### ASPECTS

Aspect 1. A hydrogel comprising a copolymer, wherein the copolymer comprises at least one residue from (a) an acrylamide selected from the group consisting of a C₁-C₄ N-alkyl acrylamide, a C₁-C₄ N,N-dialkyl acrylamide, and a combination thereof and (b) an alkyl acrylate selected from the group consisting of a C₅-C₁₈ alkyl acrylate, a C₅-C₁₈ alkyl methacrylate, and a combination thereof, wherein the alkyl acrylate is at most 3 weight percent of the copolymer.

Aspect 2. The hydrogel of Aspect 1, wherein the acrylamide is selected from the group consisting of n-isopropylacrylamide, N-propylacrylamide, N-propylmethacrylamide, N-ethoxyethylacrylamide, N-tetrahydrofurfurylacrylamide, N-tetrahydrofurfurylmethacrylamide, N,N-diethylacrylamide, N-isopropylmethacrylamide, N-tert-butylacrylamide, N-ethylacrylamide, N-cyclopropylacrylamide, N,N-dimethylacrylamide, N,N-diethylacrylamide, N-ethylacrylamide, or any combination thereof.

Aspect 3. The hydrogel of Aspect 1, wherein the acrylamide is n-isopropylacrylamide.

Aspect 4. The hydrogel of any one of Aspects 1-3, wherein the alkyl acrylate is a C₅-C₁₅ alkyl acrylate.

Aspect 5. The hydrogel of any one of Aspects 1-3, wherein the alkyl acrylate is pentyl acrylate, hexyl acrylate, 2-ethylhexyl acrylate, heptyl acrylate, octyl acrylate, nonyl acrylate, decyl acrylate, undecyl acrylate, dodecyl acrylate, hexadecyl acrylate, stearyl acrylate, lauryl acrylate, isobornyl acrylate and any combination thereof.

Aspect 6. The hydrogel of Aspect 1, wherein the copolymer comprises residues of n-isopropylacrylamide and a C₅-C₁₅ alkyl acrylate.

Aspect 7. The hydrogel of Aspect 1, wherein the copolymer consists essentially of residues of the acrylamide and the alkyl acrylate.

Aspect 8. The hydrogel of Aspect 1, wherein the copolymer consists of residues of the acrylamide and the alkyl acrylate.

Aspect 9. The hydrogel of Aspect 1, wherein the copolymer comprises residues of n-isopropylacrylamide and a C₅-C₁₅ alkyl acrylate.

Aspect 10. The hydrogel of Aspect 1, wherein the copolymer consists essentially of residues of n-isopropylacrylamide and a C₅-C₁₅ alkyl acrylate.

Aspect 11. The hydrogel of Aspect 1, wherein the copolymer consists of residues of n-isopropylacrylamide and a C₅-C₁₅ alkyl acrylate.

Aspect 12. The hydrogel of any one of Aspects 6-11, wherein the C₅-C₁₅ alkyl acrylate is selected from the group consisting of hexyl acrylate, heptyl acrylate, octyl acrylate, ethyl hexyl acrylate, dodecyl acrylate, stearyl acrylate, and any combination thereof.

Aspect 13. The hydrogel of any one of Aspects 1-12, wherein the alkyl acrylate is from about 0.05 weight percent to about 1.5 weight percent of the copolymer.

Aspect 14. The hydrogel of any one of Aspects 1-13, wherein the copolymer is covalently crosslinked.

Aspect 15. The hydrogel of Aspect 14, wherein the copolymer is covalently crosslinked with a bis-acrylamide compound or a bis-acrylate compound.

Aspect 16. The hydrogel of Aspect 14, wherein the covalent crosslinker is selected from the group consisting of methylenebisacrylamide, polyethyleneglycol diacrylate, ethylene glycol dimethacrylate, N-(1-hydroxy-2,2-dimethoxyethyl)acrylamide, divinylbenzene, phenylenediacrylamide, diurethane dimethacrylate, 1,1,1-trimethylolpropane triacrylate, 1,1,1-trimethylolpropane trimethacrylate, pentaerythritol tetraacrylate, dipentaerythritol pentaacrylate, and any combination thereof.

Aspect 17. The hydrogel of any one of Aspects 1-16, wherein the copolymer further comprises one or more residues comprising a cleavable group.

Aspect 18. The hydrogel of Aspect 17, wherein the cleavable group is selected from the group consisting of an arylsulfate, a disulfide, a peptide bond, a hydrazone, an acetyl group, a nitrobenzyl group, coumarin, an azide-alkyne, a thiolene group, or a maleimide.

Aspect 19. The hydrogel of Aspects 17 or 18, wherein a pharmaceutical compound or therapeutic compound is bonded to the cleavable group.

Aspect 20. The hydrogel of any one of Aspects 1-19, wherein the copolymer has a number average molecular weight of about 10,000 to about 300,000 daltons.

Aspect 21. The hydrogel of any one of Aspects 1-20, wherein the hydrogel further comprises at least one excipient.

Aspect 22. The hydrogel of any one of Aspects 1-21, wherein the hydrogel further comprises at least one additive.

Aspect 23. The hydrogel of any one of Aspects 1-22, wherein the copolymer has a concentration of about 10 weight percent to about 60 weight percent of the hydrogel.

Aspect 24. The hydrogel of any one of Aspects 1-23, wherein the copolymer has a lower critical solution temperature from about 20° C. to about 35° C. in aqueous solution.

Aspect 25. The hydrogel of any one of Aspects 1-24, wherein the hydrogel transitions to a solid or semi-solid in physiological conditions.

Aspect 26. A method for occluding a duct or channel in a subject, the method comprising injecting the hydrogel of any one of Aspects 1-25 into the duct or channel of the subject, wherein the hydrogel is converted to a solid or semi-solid in the duct or channel to form a plug.

Aspect 27. A method for occluding a tear duct in a subject, the method comprising injecting the hydrogel of any one of Aspects 1-25 into the tear duct of the subject, wherein the hydrogel is converted to a solid or semi-solid in the tear duct to form a plug.

Aspect 28. The method of Aspect 26 or 27, wherein the plug is subsequently removed from the tear duct by applying a stimulus to the plug so that the viscosity of the stimuli-responsive polymer in the plug decreases.

Aspect 29. The method of Aspect 26 or 27, wherein the plug is removed by exposure to a fluid.

Aspect 30. The method of Aspect 29, wherein the fluid is irrigated through the tear duct such that it contacts the hydrogel.

Aspect 31. The method of Aspect 29, wherein the fluid comprises water, alkaline solution, acidic solution, buffer solution, saline, oil, or a mixture thereof.

Aspect 32. The method of Aspect 29, wherein the fluid is below the transition temperature of the hydrogel, or the LCST of the stimuli-responsive polymer.

Aspect 33. The method of Aspect 29, wherein the plug dissolves or partially dissolves in the fluid.

Aspect 34. The method of Aspect 26 or 27, wherein the plug is removed by mechanical probing, aspiration, or application of pressure.

Aspect 35. The method of any one of Aspects 27-34, wherein the occlusion of the tear duct treats an ocular disease or condition comprising dry eye disease, glaucoma, infection, punctal stenosis, a corneal lesion, post-surgical inflammation, post-surgical discomfort, or tear film instability.

Aspect 36. The method of any one of Aspects 27-34, wherein the occlusion of the tear duct treats an ocular symptom comprising inflammation, pain, ocular discomfort, insufficient tear production, excessive tearing, rapid tear evaporation, light sensitivity, blurred vision, contact lens discomfort, or redness.

Aspect 37. The method of any one of Aspects 27-34, wherein the occlusion of the tear duct increases the retention of a topical pharmaceutical, wherein the topical pharmaceutical is administered prior to or after the occlusion of the tear duct.

Aspect 38. The method of any one of Aspects 27-37, wherein the tear duct comprises the puncta, the canaliculi, the lacrimal sac, or the nasolacrimal duct.

Aspect 39. The method of any one of Aspects 27-38, wherein said method increases tear moisture or volume in the eye.

Aspect 40. The method of any one of Aspects 26-39, further comprising maintaining the copolymer at a temperature at least approximately below the lower critical solution temperature of the copolymer prior to administering to the subject.

Aspect 41. The method of any one of Aspects 26-40, wherein said subject is human or an animal.

Aspect 42. The method of any one of Aspects 26-41, further comprising inserting a dilator into the duct or channel and dilating the duct or channel prior to administering to the subject.

Aspect 43. A method for treating dry eye in a subject, the method comprising
(a) injecting a hydrogel comprising a stimuli-responsive copolymer, wherein the copolymer comprises at N-isopropylacrylamide and an alkyl acrylate selected from the group consisting of a C5-C18 alkyl acrylate, wherein the alkyl acrylate is at most 3 weight percent of the copolymer;
(b) permitting said hydrogel to conform to the shape of the tear duct;
(c) activating said stimuli-responsive polymer in the hydrogel with a trigger, wherein said trigger initiates a phase transition of said stimuli-responsive polymer from a liquid or semi-liquid to a solid or semi-solid, thereby forming a plug in the tear duct to treat said dry eye in the subject; and
(d) removing the plug after treatment of said dry eye by applying a stimulus to the plug so that the viscosity of the hydrogel decreases.

Aspect 44. The method of Aspect 43, wherein the alkyl acrylate is pentyl acrylate, hexyl acrylate, 2-ethylhexyl acrylate, heptyl acrylate, octyl acrylate, nonyl acrylate, decyl acrylate, undecyl acrylate, dodecyl acrylate, hexadecyl acrylate, stearyl acrylate, lauryl acrylate, isobornyl acrylate and any combination thereof.

Aspect 45. The method of Aspect 43, wherein the copolymer consists of residues of n-isopropylacrylamide and a C5-C18 alkyl acrylate.

Aspect 46. The method of Aspect 43, wherein the alkyl acrylate is from about 0.02 weight percent to about 2 weight percent of the copolymer.

Aspect 47. The method of Aspect 43, wherein prior to step (a), the hydrogel is subjected to electron beam radiation.

Aspect 48. The method of Aspect 47, wherein the hydrogel is subjected to electron beam radiation at a total dosage between about 5 kGY and about 45 kGY.

Aspect 49. The method of Aspect 47, wherein the hydrogel is subjected to electron beam radiation in a single dose or in two or more sequential doses.

Aspect 50. The method of Aspect 47, wherein prior to subjecting the hydrogel to electron beam radiation, pre-conditioning the hydrogel to a temperature between −20° C. and 10° C. prior to being subjected to electron beam radiation.

Aspect 51. The method of Aspect 43, wherein the copolymer further comprises one or more residues comprising a cleavable group.

Aspect 52. The method of Aspect 51, wherein the cleavable group is selected from the group consisting of an arylsulfate, a disulfide, a peptide bond, a hydrazone, an acetyl group, a nitrobenzyl group, coumarin, an azide-alkyne, a thiolene group, or a maleimide.

Aspect 53. The method of Aspect 51, wherein a pharmaceutical compound or therapeutic compound is bonded to the cleavable group.

Aspect 54. The method of Aspect 43, wherein the copolymer has a number average molecular weight of about 10,000 to about 300,000 daltons.

Aspect 55. The method of Aspect 43, wherein the hydrogel further comprises at least one excipient.

Aspect 56. The method of Aspect 43, wherein the hydrogel further comprises at least one additive.

Aspect 57. The method of Aspect 43, wherein the trigger in step (c) comprises heating applied from said subject's body temperature to form the plug.

Aspect 58. The method of Aspect 43, wherein in step (d), the plug is contacted with a fluid to remove the plug.

Aspect 59. The method of Aspect 43, wherein the plug in step (d) is exposed to a fluid having a temperature less than 15° C.

Aspect 60. The method of Aspect 43, wherein the method further treats an ocular disease or condition comprising glaucoma, infection, punctal stenosis, a corneal lesion, post-surgical inflammation, post-surgical discomfort, or tear film instability.

Aspect 61. The method of Aspect 43, wherein the method further treats an ocular symptom comprising inflammation, pain, ocular discomfort, insufficient tear production, excessive tearing, rapid tear evaporation, light sensitivity, blurred vision, contact lens discomfort, or redness.

Aspect 62. The method of Aspect 43, wherein the method further comprises administering a topical pharmaceutical prior to step (a), after step (a) or a combination thereof.

### EXAMPLES

The following examples are put forth so as to provide those of ordinary skill in the art with a complete disclosure and description of how the compounds, compositions, and methods described and claimed herein are made and evaluated and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, the temperature is in ° C. or is at ambient temperature, and pressure is at or near atmospheric. Numerous variations and combinations of reaction conditions (e.g., component concentrations, desired solvents, solvent mixtures, temperatures, pressures, and other reaction ranges and conditions) can be used to further optimize the reagent consumption while at the same time increase the extraction efficiency.

### Example 1. Modification of Hydrogel Properties

When used in the appropriate solvent, the copolymer allows for tailored mechanical properties within a range of possible smart behavior trigger points. Using the example of a hydrogel with a cloud point temperature, an N-isopropylacrylamide (NIPAM) copolymer may have desirable mechanical properties, but a cloud point (Tcp) that renders its formulation unusable for a specific application. Examples of these effects are shown in Tables 1 and 2. This cloud point may be further affected by the excipients included in the formulation through well document phenomena.

TABLE 1
N-Isopropylacrylamide/Butyl Acrylate Copolymers

| NIPAM | BA | Tcp ° C. |
|---|---|---|
| 100% | 0% | 32 |
| 99% | 1% | 29 |
| 98% | 2% | 27.5 |
| 97% | 3% | 26 |
| 95% | 5% | 18 |

TABLE 2
Binary Copolymers

| | |
|---|---|
| NIPAM | Tcp ° C. |

Dodecyl Acrylate

| | | |
|---|---|---|
| 99.9% | 0.1% | 31.5 |
| 99% | 1% | 28.5 |

Ethylhexyl Acrylate

| | | |
|---|---|---|
| 99% | 1% | 28.5 |

Stearyl Acrylate

| | | |
|---|---|---|
| 99% | 1% | 28 |
| 99.9% | 0.1% | 30.5 |

### Example 2. Pre-Conditioning of Hydrogel and Exposure to Electron Beam Radiation

Hydrogel samples are provided in standard packaging and subjected to a controlled irradiation process to induce cross-linking while preventing undesirable thermal rearrangement. Prior to irradiation, the samples are cooled to a temperature within the range of 2° C. to 8° C. using a temperature-controlled cooling unit. The samples are then transferred to the irradiation chamber, where a total dose of 38.5±2 kGy is administered in three sequential passes as follows: Pass 1: 12.8 kGy, Pass 2: 12.8 kGy, Pass 3: 12.9 kGy. Following each irradiation pass, the samples are returned to the cooling unit and allowed to equilibrate to a temperature within the 2° C. to 8° C. range before the next pass. This step ensures that the cumulative heat generated during irradiation (approximately 1° F. per kGy) does not raise the internal temperature above the critical threshold of 28° C. Upon completion of the third pass, the samples are cooled again to stabilize the hydrogel structure.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed:

1. A method for treating dry eye in a subject, the method comprising
(a) injecting a hydrogel comprising a stimuli-responsive copolymer, wherein the copolymer comprises at N-isopropylacrylamide and an alkyl acrylate selected from the group consisting of a C5-C18 alkyl acrylate, wherein the alkyl acrylate is at most 3 weight percent of the copolymer;
(b) permitting said hydrogel to conform to the shape of the tear duct;
(c) activating said stimuli-responsive polymer in the hydrogel with a trigger, wherein said trigger initiates a phase transition of said stimuli-responsive polymer from a liquid or semi-liquid to a solid or semi-solid, thereby forming a plug in the tear duct to treat said dry eye in the subject; and
(d) removing the plug after treatment of said dry eye by applying a stimulus to the plug so that the viscosity of the hydrogel decreases.

2. The method of claim 1, wherein the alkyl acrylate is pentyl acrylate, hexyl acrylate, 2-ethylhexyl acrylate, heptyl acrylate, octyl acrylate, nonyl acrylate, decyl acrylate, undecyl acrylate, dodecyl acrylate, hexadecyl acrylate, stearyl acrylate, lauryl acrylate, isobornyl acrylate and any combination thereof.

3. The method of claim 1, wherein the copolymer consists of residues of n-isopropylacrylamide and a C5-C18 alkyl acrylate.

4. The method of claim 1, wherein the alkyl acrylate is from about 0.02 weight percent to about 2 weight percent of the copolymer.

5. The method of claim 1, wherein prior to step (a), the hydrogel is subjected to electron beam radiation.

6. The method of claim 5, wherein the hydrogel is subjected to electron beam radiation at a total dosage between about 5 kGY and about 45 kGY.

7. The method of claim 5, wherein the hydrogel is subjected to electron beam radiation in a single dose or in two or more sequential doses.

8. The method of claim 5, wherein prior to subjecting the hydrogel to electron beam radiation, pre-conditioning the hydrogel to a temperature between −20° C. and 10° C. prior to being subjected to electron beam radiation.

9. The method of claim 1, wherein the copolymer further comprises one or more residues comprising a cleavable group.

10. The method of claim 9, wherein the cleavable group is selected from the group consisting of an arylsulfate, a disulfide, a peptide bond, a hydrazone, an acetyl group, a nitrobenzyl group, coumarin, an azide-alkyne, a thiolene group, or a maleimide.

11. The method of claim 9, wherein a pharmaceutical compound or therapeutic compound is bonded to the cleavable group.

12. The method of claim 1, wherein the copolymer has a number average molecular weight of about 10,000 to about 300,000 daltons.

13. The method of claim 1, wherein the hydrogel further comprises at least one excipient.

14. The method of claim 1, wherein the hydrogel further comprises at least one additive.

15. The method of claim 1, wherein the trigger in step (c) comprises heating applied from said subject's body temperature to form the plug.

16. The method of claim 1, wherein in step (d), the plug is contacted with a fluid to remove the plug.

17. The method of claim 1, wherein the plug in step (d) is exposed to a fluid having a temperature less than 15° C.

18. The method of claim 1, wherein the method further treats an ocular disease or condition comprising glaucoma, infection, punctal stenosis, a corneal lesion, post-surgical inflammation, post-surgical discomfort, or tear film instability.

19. The method of claim 1, wherein the method further treats an ocular symptom comprising inflammation, pain, ocular discomfort, insufficient tear production, excessive tearing, rapid tear evaporation, light sensitivity, blurred vision, contact lens discomfort, or redness.

20. The method of claim 1, wherein the method further comprises administering a topical pharmaceutical prior to step (a), after step (a) or a combination thereof.
